(19) United States
(12) Patent Application Publication
(10) Pub. No.: US 2010/0152787 A1
(43) Pub. Date: Jun. 17, 2010

(54) SPINAL FIXATION ASSEMBLY

(75) Inventors: David A. Walsh, Reading, MA (US); Carl Arthur Knobloch, Oviedo, FL (US); Daniel R. Baker, Seattle, WA (US)

Correspondence Address: RENNER OTTO BOISSELLE & SKLAR, LLP, 1621 EUCLID AVENUE, NINETEENTH FLOOR, CLEVELAND, OH, 44115, US

(73) Assignee: Biotechni America Spine Group, Inc., Palm Harbor, FL (US)

(21) Appl. No.: 11/816,806

(22) PCT Filed: Jul. 26, 2007
(86) PCT No.: PCT/US07/74455

§ 371 Date: Feb. 5, 2010

(51) Int. Cl. A61B 17/86

(52) U.S. Cl. 606/308; 606/305

(57) ABSTRACT

A locking mechanism (100) and method of fixation, such as the fixation of a fixation device (104) like a bone screw and of a rod (106) to the spine. The locking mechanism (100) includes a body (102), an insert (108, 308), a rod seat (110, 310) and a set screw. The body (102) includes a bottom portion (114) configured to receive the fixation device (104) and the insert (108, 308) but prevents the insert (108, 308) and fixation device (104) from passing therethrough once the insert (108, 308) and fixation device (104) are engaged. The body (102) further includes a side portion (120) configured to receive the rod (106). Between the rod (106) and the insert (108, 308) is a rod seat (110, 310).

### FIELD OF THE INVENTION

The present invention relates generally to prostheses for treating spinal pathologies, and more specifically to spinal fixation assemblies including an anchor for holding a fixation device and a stabilization rod.

### BACKGROUND OF THE INVENTION

Various methods of spinal immobilization have been used in the treatment of spinal instability and displacement. The most common treatment for spinal stabilization is immobilization of the joint by surgical fusion, or arthrodesis. This has been known for almost a century. In many cases, however, pseudoarthrosis occurs, particularly in cases involving fusion across the lumbosacral articulation and when more than two vertebrae are fused together. Early in the century, post operative external immobilization, such as through the use of splints and casts, was the favored method of spinal fixation. As surgical techniques became more sophisticated, various new methods of internal and external fixation were developed.

Internal fixation refers to therapeutic methods of stabilization that arc wholly internal to the patient and include commonly known devices such as bone plates, screws, rods and pins. External fixation, in contrast, involves at least some portion of the stabilization device being located external to the patients' body. As surgical technologies and procedures became more advanced and the likelihood of infection decreased, internal fixation eventually became the favored method of immobilization since it is less restrictive on the patient.

Internal fixation of the spine may be used to treat a variety of disorders including kyphosis, spondylolisthesis and rotation, segmental instability, such as disc degeneration and/or fracture caused by disease, trauma, congenital defects and tumor diseases. One of the main challenges associated with internal spinal fixation is securing the fixation device to the spine without damaging the spinal cord. The pedicles of a vertebra are commonly used for fixation as they generally offer an area that is strong enough to hold the fixation device in place even when the patient suffers from degenerative instability such as osteoporosis.

Current fixation devices and hardware systems used internally for spinal fixation in modern surgical procedures are generally designed to meet one or more criteria, such as: providing rigidity as is indicated, generally along the long axis of the patient's spine; accommodating a broad variation in the size and shape of the spinal member with which it is used; having the capability of handling the stresses and strains to which the devices will be subjected resulting from movement of the spine; and providing easy surgical access during both implantation and removal of the implant.

The present invention includes a novel fixation device.

### BRIEF SUMMARY OF THE INVENTION

Disclosed is a locking mechanism that includes a body having a bottom portion and a side portion, where the side portion is configured to receive a rod. The locking mechanism also includes a fixation device extending at least partially through a hole in the bottom portion of the body. The fixation device has an at least partially spherical head. In addition, the locking mechanism includes an at least partially spherical insert at least partially surrounding the head of the fixation device and a rod seat having an opening for engaging a top portion of the insert such that upon engagement, the rod seat applies forces to the insert that have both lateral and vertical components.

Also disclosed is a locking mechanism that includes a body having a bottom portion and a side portion where the side portion is configured to receive a rod. The locking mechanism further includes a fixation device extending at least partially through a hole in the bottom portion of the body where the fixation device has an at least partially spherical head. The locking mechanism also has an at least partially spherical insert at least partially surrounding the head of the fixation device such that when the insert and fixation device are forced toward the bottom of the body, the insert applies generally perpendicular force to at least one location on a surface of an upper hemisphere of the head and further applies generally perpendicular force to at least one location on a surface of a lower hemisphere of the head.

Also disclosed is a locking mechanism including a body having a bottom portion and a side portion where the side portion is configured to receive a rod selected from a group of rods of varying diameter. The locking mechanism also includes a fixation device extending at least partially through a hole in the bottom portion of the body. Between the rod and the fixation device is a rod seat that has a taper for engaging the selected rod and aligning the center of the selected rod with the central axis of the body, where the taper includes two sides and at least two curvatures on each side, and where each curvature has a surface for engaging the selected rod such that the diameter of the selected rod determines which of the surfaces of the at least two curvatures engages the selected rod.

Further disclosed is a locking mechanism that includes a body having a bottom portion and a side portion where the side portion is configured to receive a rod. The locking mechanism further includes a fixation device having a spherical head and extending at least partially through a hole in the bottom portion of the body. An at least partially spherical insert at least partially surrounds the head of the fixation device. The insert has downward fingers extending from a portion of the insert corresponding to an upper hemisphere of the head of the fixation device to a portion of the insert corresponding to a lower hemisphere of the head of the fixation device and upward fingers extending from the portion of the insert corresponding to the lower hemisphere of the head of the fixation device to the portion of the insert corresponding to the upper hemisphere of the head of the fixation device. The locking mechanism also includes a rod seat having a tapered portion for receiving the rod and an opening for engaging a top portion of the insert such that upon engagement, the rod seat applies forces to the insert that have both lateral and vertical components.

Further disclosed is a method for locking the relative positions of a fixation device and a rod. The method includes placing an insert through a hole in a bottom portion of a body of a locking mechanism; placing a fixation device having a generally spherical head through the hole in the bottom portion of the body of the locking mechanism; engaging the head of the fixation device with the insert; engaging a top portion of the insert with a rod seat; placing the rod in a side portion of the body such that the rod and insert are on opposite sides of the rod seat; and urging the rod toward the rod seat such that the rod seat applies forces to the insert that have both lateral and vertical components.

Also disclosed is a method for locking the relative positions of a fixation device and a rod. The method includes placing an insert through a hole in a bottom portion of a body of a locking mechanism; placing a fixation device having a generally spherical head through the hole in the bottom portion of the body of the locking mechanism; engaging the head of the fixation device with the insert such that the insert at least partially surrounds the head of the fixation device; placing the rod in a side portion of the body such that the rod and the insert bottom portion of the body are on opposite sides of the insert; and causing the insert to apply generally perpendicular force to at least one location on a surface of an upper hemisphere of the head of the fixation device and further to apply generally perpendicular force to at least one location on a surface of a lower hemisphere of the head of the fixation device.

The features of the present invention will be apparent with reference to the following description and attached drawings. In the description and drawings, particular embodiments of the invention have been disclosed in detail as being indicative of some of the ways in which the principles of the invention may be employed, but it is understood that the invention is not limited correspondingly in scope. Rather, the invention includes all changes, modifications and equivalents coming within the spirit and terms of the claims appended hereto.

Features that are described and/or illustrated with respect to one embodiment may be used in the same way or in a similar way in one or more other embodiments and/or in combination with or instead of the features of the other embodiments.

### BRIEF DESCRIPTION OF THE SEVERAL VIEWS OF THE DRAWINGS

FIG. 1 is an exploded perspective view of a locking mechanism of the present invention with a fixation device and a rod;

FIG. 2A is a cross-sectional view of the locking mechanism of the present invention in an unlocked position;

FIG. 2B is a cross-sectional view of the locking mechanism of the present invention in a locked position;

FIG. 3A is a cross-sectional view of an alternate embodiment of the locking mechanism of the present invention in an unlocked position;

FIG. 3B is a cross-sectional view of an alternate embodiment of the locking mechanism of the present invention in a locked position;

FIGS. 4A-B are top and bottom perspective views of the body of the locking mechanism of the present invention;

FIGS. 5A-B are top and bottom perspective views of the insert of FIGS. 2A and 2B;

FIGS. 6A-B are top and bottom perspective views of the rod seat of FIGS. 2A and 2B;

FIGS. 7A-B are top and bottom perspective views of the insert of FIGS. 3A and 3B;

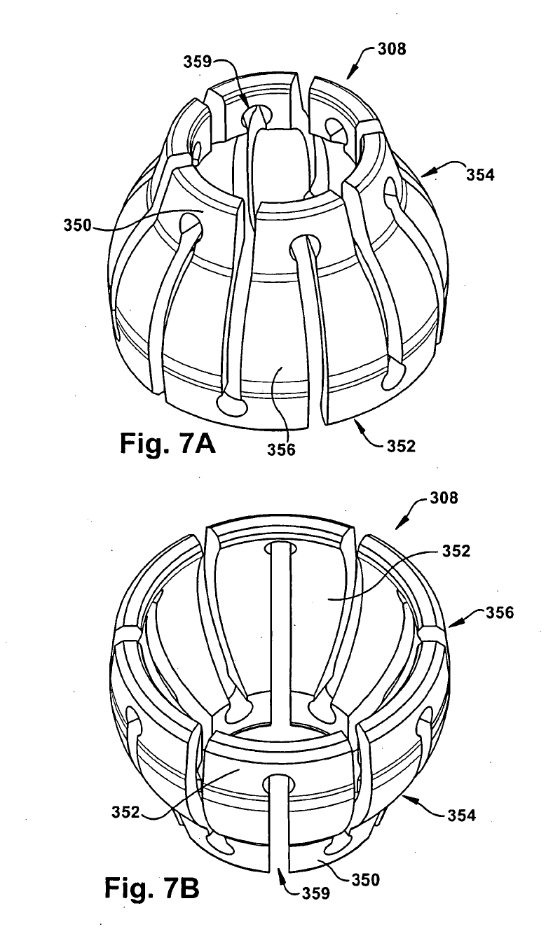

FIGS. 8A-B are top and bottom perspective views of the rod seat of FIGS. 3A and 3B;

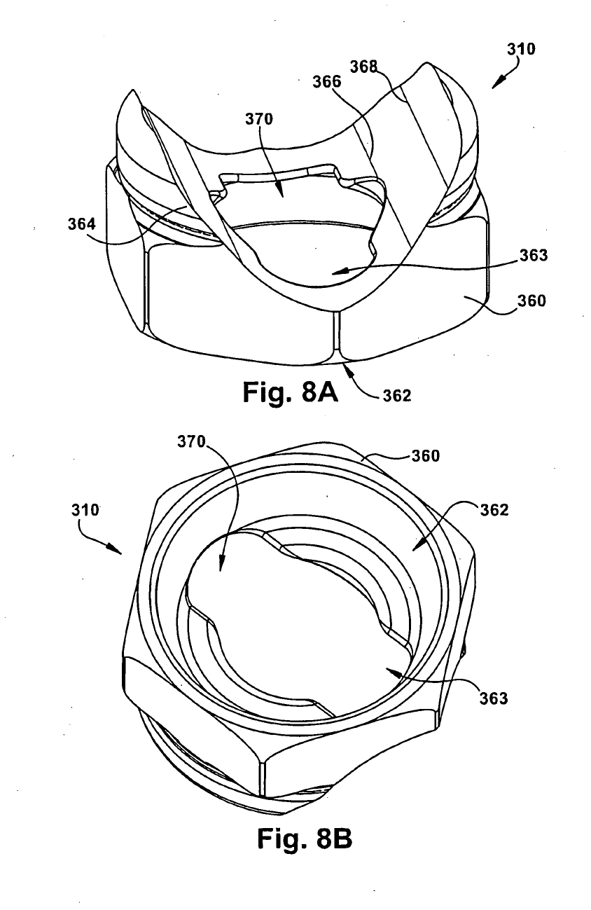

FIG. 9 is a flow chart illustrating a novel method of fixing the position of a rod with respect to a fixation device according to the present invention; and

FIG. 10 is a flow chart illustrating another novel method of fixing the position of a rod with respect to a fixation device according to the present invention.

### DETAILED DESCRIPTION OF THE INVENTION

The invention relates to a novel locking mechanism and method for locking the relative positions of a rod and a fixation device. The locking mechanism provides an improved lock between a rod and the head of a fixation device, such as a screw. The apparatus includes a body and an internal insert that at least partially surrounds the head of the fixation device. When the locking mechanism is used to lock the fixation device, the insert preferably functions to “squeeze” the head of the fixation device so that forces are applied to both the upper and lower portions of the head, thereby fixing the fixation device with respect to the body. In addition, the insert may be configured to cause forces having both lateral and vertical components to be applied to the head of the fixation device to prevent its movement.

The invention may also include a novel rod seat that applies lateral forces to the insert when compressed into the insert as the rod is urged downward in the body. In addition, the novel rod seat may be designed to provide multiple contact surface areas for rods of varying diameter while helping to position the rod along the central axis of the body as the rod is urged downward in the body.

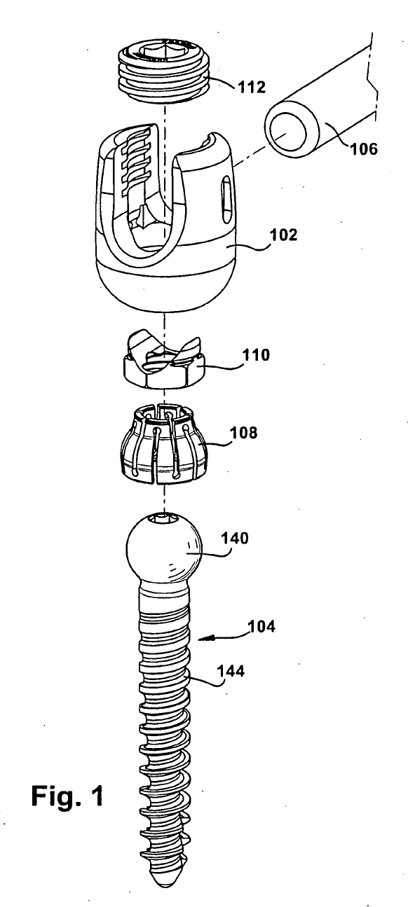

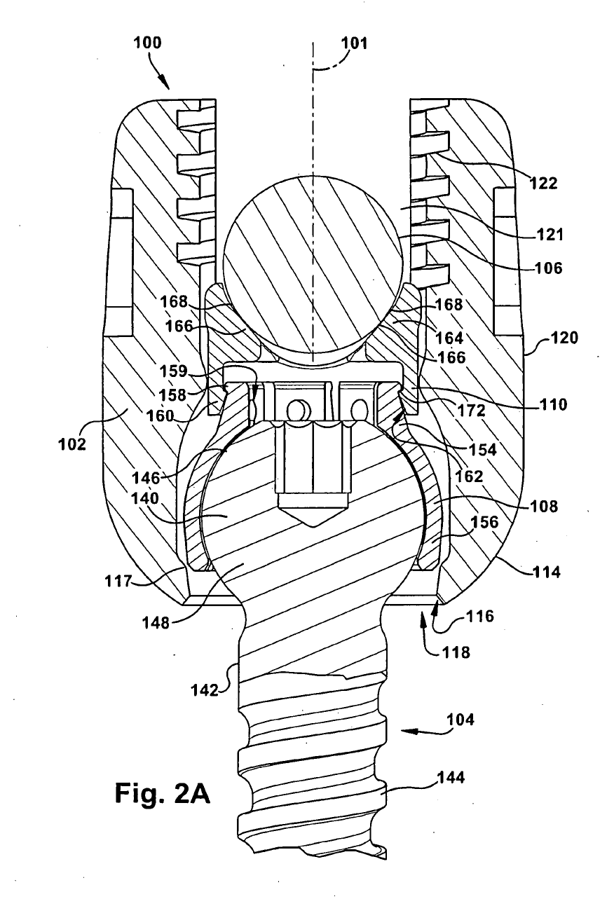

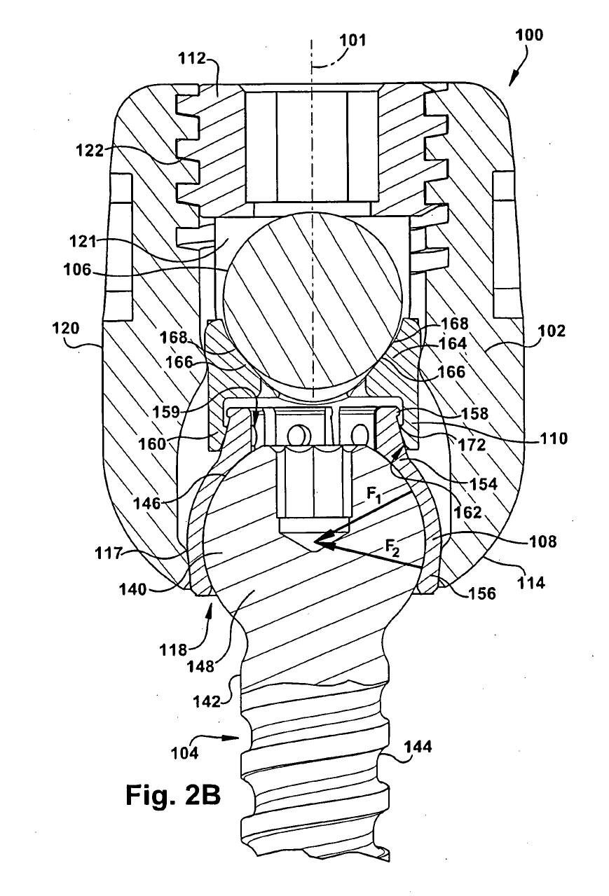

Turning initially to FIGS. 1 and 2A-B, FIG. 1 shows an exploded perspective view of a locking mechanism of the present invention, and FIGS. 2A-B show cross sectional views of the locking mechanism of FIG. 1. The locking mechanism 100 is configured to engage and lock the position of a fixation device 104 with respect to the position of a rod 106. The locking mechanism 100 includes a body 102, insert 108, rod seat 110 and locking element 112.

When the locking mechanism 100 is used for spinal fixation, “above” or “top” means posterior with respect to the patient and “below” or “bottom” means anterior with respect to the patient. Thus, the bottom portion 114 of the body 102 is anterior with respect to the patient and the rod 106 is received by the body 102 as the rod 106 is moved in a posterior to anterior direction.

The body 102 includes a bottom portion 114 that includes a hole 118 configured to receive the insert 108 and the fixation device 104 such that the socket 116 of the bottom portion 114 engages part of the insert 108 and prevents the insert 108 and fixation device 104 from exiting the body once they are inserted into the body 102 and engaged with one another. For example, the insert 108 may be compressible to enable insertion into the hole 118. Once inserted into the body 102 via the hole 118, however, the insert 108 may expand to have a width greater than that of the diameter of the hole 118.

The fixation device 104 may be, for example, a screw having a head 140 and shaft 142 with threads 144. When the fixation device 104 is inserted into the hole 118 of the body 102, the head 140 of the fixation device 104 preferably engages the insert 108 in a snap-fit manner such that the insert 108 expands to accommodate the head 140. When the insert 108 and fixation device 104 combination is forced toward the bottom portion 114 of the body 102, the lip 117 of the socket 116 of the bottom portion 114 of the body 102 engages the sides of the insert 108, causing the insert 108 to more tightly engage the head 140 of the fixation device 104 and preventing the insert 108 and the fixation device 104 from exiting the body 102 through the hole 118. When the insert 108 and head 140 are urged into contact with the socket 116, the insert 108 may exert forces on the lower hemisphere 148 of the head 140 that have both lateral and vertical components. Preferably, the insert 108 exerts a generally perpendicular force F2 to at least one location on the surface of the lower hemisphere 148 of the head 140, although it will be understood by those skilled in the art that generally perpendicular forces are not required between the insert 108 and the head 140 in order to fix the positions of the head 140 and insert 108 with respect to the body 102.

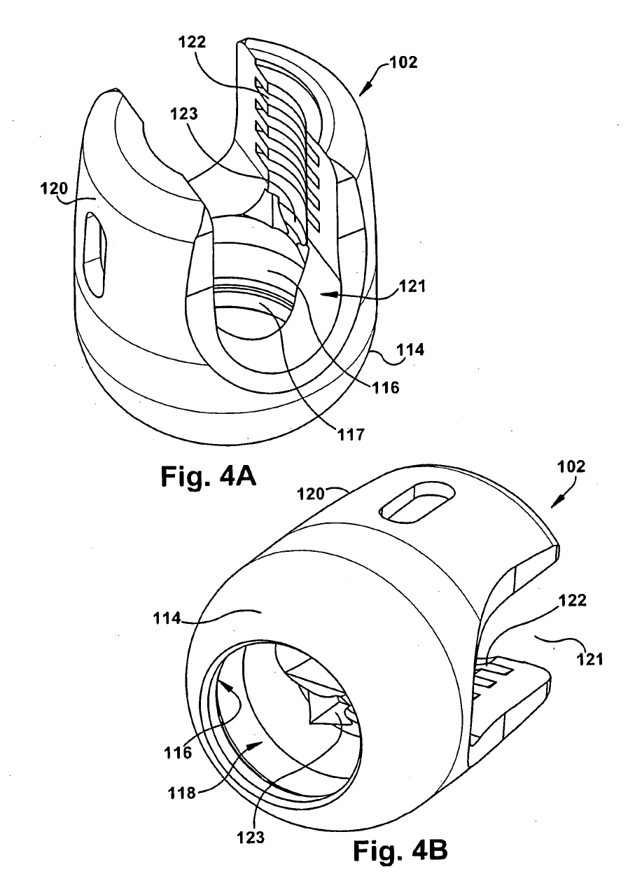

The body 102 also includes a side portion 120 that is configured to receive the rod 106, such as by way of a channel 121 that enables placement of the rod 106 by either sliding the rod 106 through the side portion 120 of the body or by inserting the rod 106 into the channel 121 through the top portion of the body 102. The body 102 is also configured to receive a rod seat 110, for example, through the hole 118 in the bottom of the body 102. It will be understood by those skilled in the art that the rod scat 110 and body 102 may also be designed such that that the rod seat 110 is configured for insertion through the top of the body 102. The rod scat 110 is preferably inserted into the body 102 prior to insertion of the rod 106 such that the rod seat 110 is eventually positioned between the rod 106 and the insert 108. The body 102 also preferably includes an alignment channel 123 (FIGS. 4A-B) for engaging the rod seat 110 and maintaining proper alignment of the rod seat 110 within the body 102. Likewise, the rod seat 110 is preferably configured to engage the alignment channel 123 when placed in the body 102.

The rod seat 110 has a tapered portion 164 for receiving the rod 106. Preferably, the center of the tapered portion 164 is aligned with the central axis 101 of the body to facilitate alignment of the rod 106 within the body 102. The tapered portion 164 of the rod seat 110 may also be configured to engage rods of varying diameters, such as rods having diameters ranging from 3 mm to 7 mm. For example, the tapered portion 164 of the rod seat 110 may have multiple curvatures on each side of the rod seat 110, such as curvatures 166 and 168 that provide varying surfaces for contacting rods of varying diameters.

When the rod seat 110 is forced downward by the rod 106, the rod seat 110 may exert forces on the insert 108 that have both lateral and vertical components. This in turn may cause the insert 108 to exert forces that have both lateral and vertical components on the upper hemisphere 146 of the head 140. Preferably, such movement causes the insert 108 to exerts a generally perpendicular force F1 to at least one location on the surface of the upper hemisphere 146 of the head 140, although it will be understood by those skilled in the art that generally perpendicular forces are not required between the insert 108 and the head 140 in order to fix the positions of the head 140 and insert 108 with respect to the body 102,

The locking mechanism 100 may also include a locking element 112 that is configured to engage the body 102 and the rod 106 so as to force the rod 106 toward the fixation device 104. The locking element 112 and body 102 may be, for example, slidably engageable, rotatably engageable and/or snapably engageable. Accordingly, the body 102 may include threads 122 for engaging the locking element 112. In addition, while FIGS. 1 and 2A-B show internal threads, it will be understood by those of ordinary skill in the art that the threads may also be external threads and the locking element 112 may surround the body 102 during engagement. Preferably, the locking element 112 is a set screw, which may be either internally or externally threaded to engage either an externally or internally threaded body 102.

Turning now to FIGS. 2A and 2B, the locking mechanism 100 of the present invention is illustrated in greater detail. FIG. 2A is a cross-sectional view of the locking mechanism 100 of the present invention in an unlocked position. FIG. 2B is a cross-sectional view of the locking mechanism 100 of the present invention in a locked position.

The locking mechanism 100 includes a body 102, an insert 108, a rod seat 110 and a locking element 112 for engaging a fixation device 104 and a rod 106. Each of the body 102, the insert 108, the rod seat 110, the locking element 112, the fixation device 104 and the rod 106 may be made from a variety of materials known in the art and preferably is made from a biocompatible material when the locking mechanism 100 is used for bone fixation. Such materials include, but are not limited to, titanium, titanium alloys (e.g. titanium/aluminum/vanadium (Ti/Al/V) alloys), cobalt-chromium alloys, stainless steel, ceramics (alumina ceramic, zirconia ceramic, yttria zirconia ceramic, etc.), high strength polymers (e.g. PEEK, PEKK, etc.), pyrolytic carbon, tantalum, carbon composite materials and combinations thereof, which may include mechanically compatible mixtures of the above materials. Such materials are commonly used in bone fixation and the like. Preferably, the materials are rigid and in one embodiment, the body 102, fixation device 104, rod 106, insert 108, rod seat 110 and locking element 112 are all made from Ti/Al/V alloys, such as Ti/6Al/4V ELI.

While one of skill in the art will recognize that fixation devices 104 other than a screw can be used without departing from the scope of the present invention, a screw is shown and described herein to illustrate the engagement of the fixation device 104 and the body 102, as well as the method for locking the relative positions of a fixation device 104 and a rod 106. Furthermore, various types of screws may be used. Preferably, however, the fixation device 104 has an at least partially spherical head 140.

The size of the body 102 may be similar to that of known devices. For example, the height of body 102 may range from about 0.4 inch to about 1 inch. Also, the width of body 102 may range from about 0.25 inch to about 1 inch.

The body 102 has a side portion 120 and a bottom portion 114. The bottom portion 114 may be tapered and may have a socket 116 and a hole 118. Because the general shape of the type of body 102 illustrated in FIGS. 1, 2A-B and 4A-B somewhat resembles a tulip flower, the type of body 102 is often referred to as a “tulip” by those skilled in the art. The socket 116 is preferably sized to accept the head 140 of the fixation device 104, such as a screw. The hole 118 is preferably located at the bottom of the body 102.

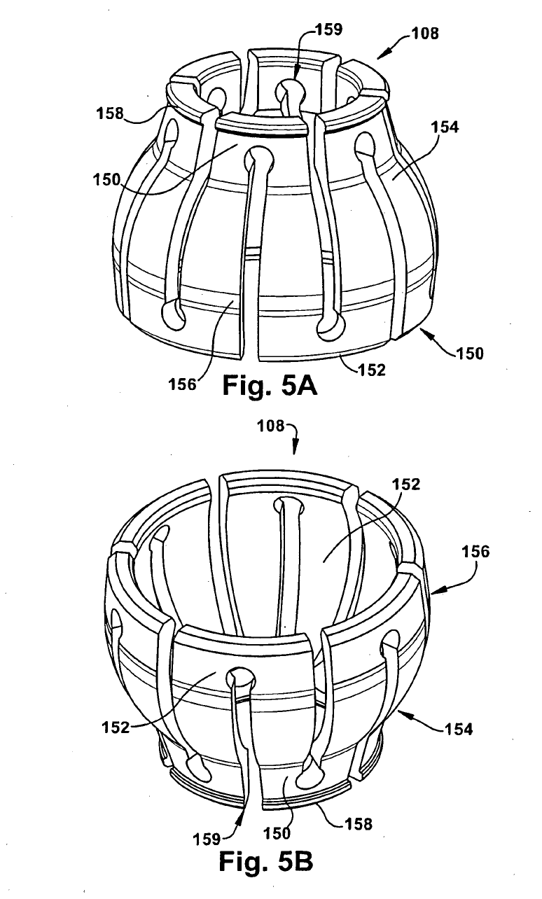

Housed within the body 102 is an insert 108, shown in greater detail in FIGS. 5A-B. The insert 108 is preferably compressible so that it can be inserted through the hole 118 in the bottom of the body 102 when in a compressed state. The internal portion of the body 102 is preferably large enough that the insert 108 can return to an uncompressed state after being placed within the body 102 via the hole 118. As illustrated in greater detail in FIGS. 5A-B. Preferably, the insert 108 is at least partially spherical and has an upper hemisphere portion 154 and a lower hemisphere portion 156. Accordingly, the diameter of the at least partially spherical section of the insert 108 is preferably larger than the hole 118 when the insert 108 is in an uncompressed state and preferably smaller than the hole 118 when the insert 108 is in a compressed state.

To facilitate compression and engagement with the head 140 of the fixation device 104, the insert 108 may include fingers, such as upward and downward interdigitating fingers. As shown in FIGS. 5A-B, the insert 108 has interdigitating upward extending fingers 150 and downward extending fingers 152. In addition, upward extending fingers 150 may extend from an upper hemisphere portion 154 of the insert 108 to a lower hemisphere portion 156 of the insert 108 and the downward extending fingers 152 may extend from the lower hemisphere portion 156 of the insert 108 to an upper hemisphere portion 154 of the insert 108. Further, the upward extending fingers 150 and the downward extending fingers 152 may or may not alternate around the perimeter of the insert 108.

Preferably, the insert 108 is at least partially spherical and is configured to at least partially surround the head 140 of the fixation device 104. The diameter of the head 140 is preferably less than that of the hole 118 so that the head 140 can be inserted into and removed from the body 102. Alternatively, the diameter of the head 140 can be larger than the hole 118 so that the head 140 could not be removed from the body 102. In this case, the body 102 is preferably formed around the insert 108 and the head 140. In the preferred embodiment, however, the insert 108 and the head 140 can both be inserted into the body 102 through the hole 118. Once inserted, the head 140 preferably engages the insert 108. When engaged, the insert 108 at least partially surrounds the head 140 such that the diameter of the insert 108 is greater than that of the hole 118. The insert 108 may be configured to snapably engage the head 140 of the fixation device 104 when the head 140 is inserted into the hole 118 of the body 102.

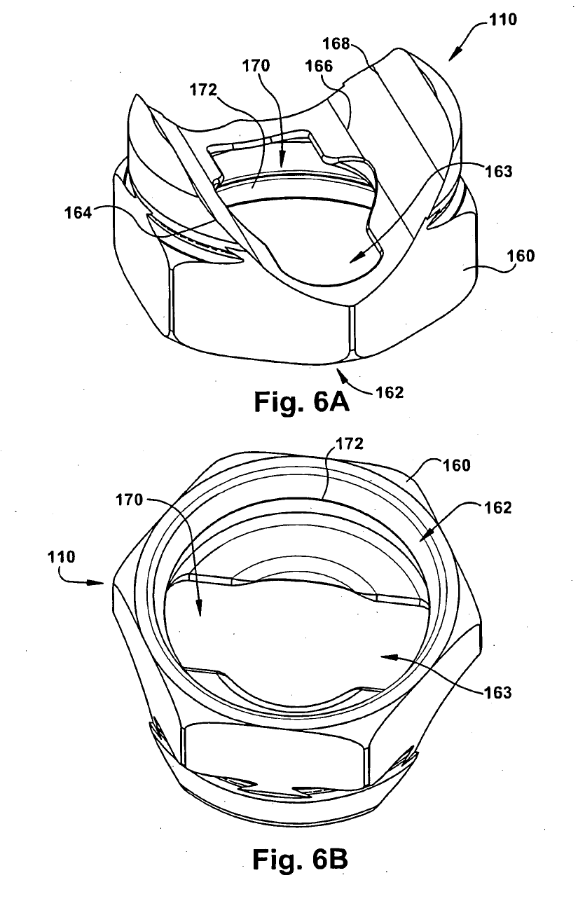

Above the insert 108 is the rod seat 110, shown in greater detail in FIGS. 6A-B, which may be insertable through the bottom of the body 102. The rod seat 110 is preferably configured for interaction with the alignment channel 123 (FIG. 4A) of the body 102. For example, as shown in FIGS. 6A-B, the rod seat 110 is non-circular and has shape that engages the contours of the interior of the body 102 to facilitate proper positioning of the rod seat 110 within the body 102.

The rod seat 110 may serve multiple functions, such as aiding in the alignment of the rod 106, aiding in the alignment of the insert 108, creating a contact surface for the rod 106, causing the insert 108 to exert forces on the head 140 that have both vertical and lateral components; facilitating the disengagement of the insert 108 from the body 102 when in a locked position, and preventing linear compressive forces from being transferred from the rod 106 to the top of the insert 108 or top of the head 140.

The rod seat 110 has a taper 164 to facilitate placement of the rod 106 within the body 102. Preferably, the midline of the taper 164 is aligned with the central axis 101 of the body 102. In this manner, the taper 164 facilitates placement of the rod 106 within the body 102 such that the center of the rod 106 is generally aligned with the central axis 101 of the body 102. Moreover, since the upper portion of the taper 164 of the rod seat 110 is preferably wider than the lower portion of the rod seat 110, the taper 164 the sides of the taper 164 are preferably forced toward the central axis 101 of the body 102 as the rod seat 110 is forced toward the bottom of the body 102, such as by compression of the rod 106 into the rod seat 110. Thus, as the rod 106 is forced downward, the taper 164 of the rod seat 110 preferably applies forces to the rod 106 that have lateral components, further facilitating the locking the rod 106 and the alignment of the rod 106 with the central axis 101 of the body 102.

In addition, the taper 164 may include multiple curvatures, such as curvatures 166 and 168 on each side of the rod scat 110. FIGS. 6A-B illustrate a rod seat 110 with two curvatures, but the taper 164 may have three or more curvatures as well. The multiple curvatures create engagement surfaces for rods of varying diameter. In the preferred embodiment, the rod seat 110 is configured to engage a rod 106 where the rod 106 has a diameter ranging between 3 mm and 7 mm. The diameter of the rod 106 may determine which of the curvatures 166 or 168 contacts the rod 106. For example, the surface of curvature 166 may be the primary engagement surface for a 3 mm rod 106, but the surface of curvature 168 may be the primary engagement surface for a 7 mm rod 106. In addition, it will be understood by those skilled in the art that the surfaces of curvatures 166 and 168 may overlap and that a rod 106 may contact the surface of both curvature 166 and curvature 168.

The rod seat 110 also preferably engages the outer surface of at least a portion of the upper hemisphere 154 of the insert 108. For example, and as shown in FIGS. 2A-B, the rod seat 110 may include a skirt 160 having a latch 172 for retaining the lip 158 of the insert 108. In this manner, the insert 108 and the rod seat 110 may be snapably engageable. The rod seat 110 also preferably includes a top hole 170 and a bottom hole 163 to provide access to the head 140 of the fixation device 104 from the top of the body 102. Similarly, the insert 108 also preferably includes an access hole 159 to the head 140. Thus, a driver or similar device may be used to engage the head 140 of the fixation device 104 via the rod seat 110 and the insert 108. The insert 108 may function as a collet to facilitate movement from the locked position of FIG. 2B to the unlocked position of FIG. 2A. In this manner, the fixation device 104 can be tightened using a driver or the like without permanently fixing the position of the fixation device 104 with respect to the body 102 and rod 106. In use, a driver can be used to tighten the fixation device 104.

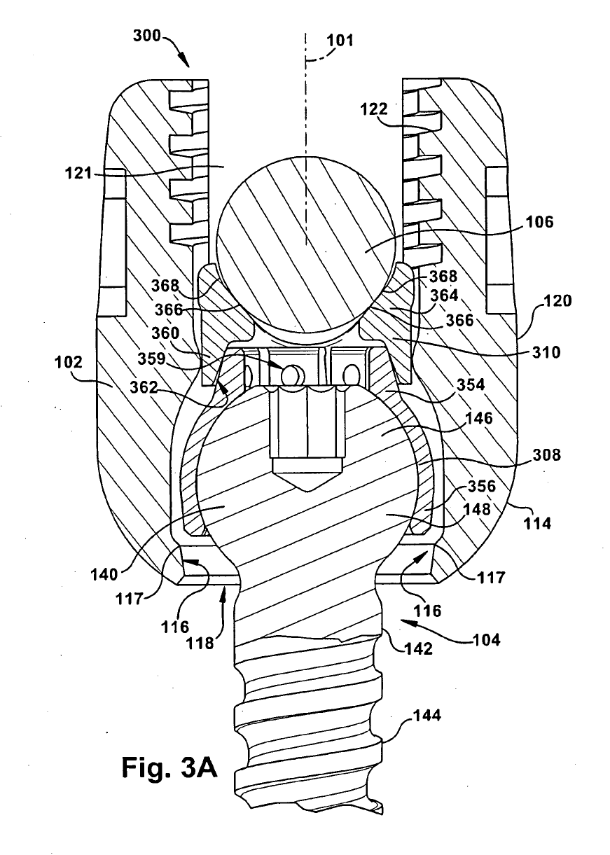

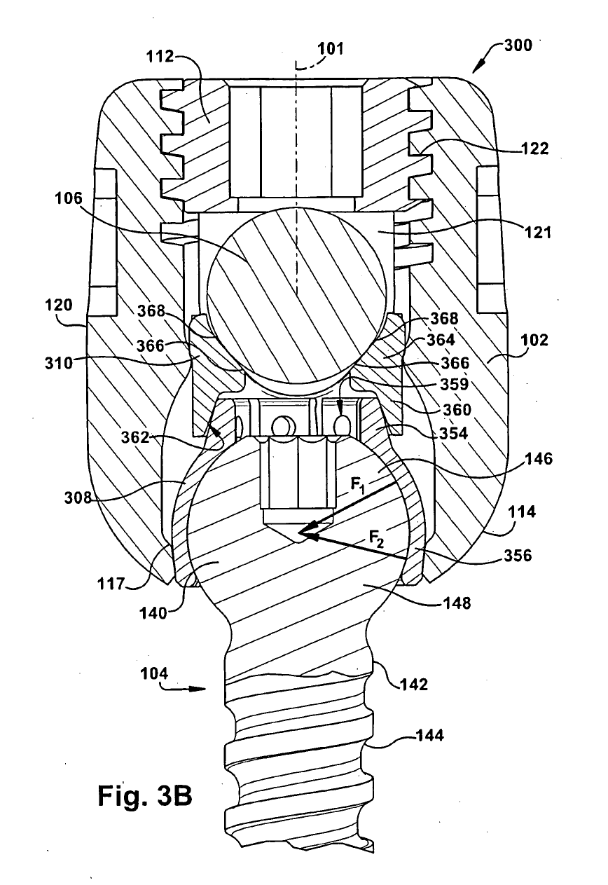

Following the tightening of the fixation device 104, it may be desirable to use the driver or other tool to pull the insert 108 and rod seat 110 into an unlocked position so that an appropriate angle can be determined for fixing the positions of the fixation device 104 and rod 106 with respect to one another. This may be accomplished by pulling the rod seat 110 upward while the latch 172 of the skirt 160 engages the lip 158 of the insert 108 and pulls the insert 108 upward. Alternatively, the movement to an unlocked position may be accomplished by directly engaging and pulling the insert 108 toward the top of the body 102. The alternate method for unlocking mechanism 100 may also be used for the locking mechanism 300 of FIGS. 3A-B.

In addition the skirt 160 may have a taper 162 that engages the insert 108 in a manner that forces a portion of the upper hemisphere 154 of the insert 108 both downward toward the bottom of the body 102 and inward toward the center of the insert 108. In turn, this may cause the insert 108 to exert forces on the upper hemisphere 146 of the head 140 that have both lateral and vertical components. Further, the interaction of the rod seat 110 and the insert 108 may cause the insert 108 to exert at least one force F1 upon the upper hemisphere 146 of the head 140 that is generally perpendicular to the surface of the upper hemisphere 146 of head 140 upon which the force F1 is exerted.

The body 102 also includes a channel 121 in the side portion 120 for receiving the rod 106 or other stabilization element, such as a dynamic stabilization element. While a channel 121 is preferred for receiving the rod 106, it will be understood by those skilled in the art that an aperture in the side portion 120 could also receive the rod 106, though a body 102 with an aperture may be more cumbersome to deploy during surgery as a surgeon would have to place the rod 106 through the aperture instead of placing the rod 106 in the channel 121. Using a channel 121 to receive the rod 106 provides greater flexibility for a surgeon.

After placement of the rod 106 within the channel 121, it is desirable to retain the rod 106 within the channel 121. Accordingly, the side portion 120 of the body 102 also includes threads 122 configured to engage locking element 112, which is preferably a set screw. Although internal threads 122 are illustrated in FIGS. 1, 2A-B and 4A-B, the threads 122 may be either internal or external to the body 102 depending on the configuration of the body 102 and the locking element 112.

The locking element 112 is thus engaged with the threads 122 of the side portion 120 of the body 102 to keep the rod 106 within the channel 121. It should be understood, however, that the locking element 112 need not engage the body 102 via threaded engagement. The locking element 112 and the body 102 may be slidably engageable, rotatably engageable, and/or snapably engageable. In the embodiment disclosed in FIGS. 1 and 2A-B the locking element 112 and the body 102 are rotatably engageable. To fix the rod 106 with respect to the fixation device 104, the locking element 112 preferably is tightened down to apply increasing force to the rod 106 in order to engage and lock the rod 106 and fixation device 104. More specifically, the tightening of the locking element 112 causes linear compression of the rod 106 onto the rod seat 110, which in turn causes the insert 108 to engage to the fixation device 104 and forces the insert 108 and the fixation device 104 toward the bottom portion 114 of the body 102.

Although the rod 106 forces the rod seat 110 downward, the manner in which the rod seat 110 engages the insert 108 may prevent the transfer of linear compressive forces from the rod seat 110 to the top of the insert 108 or to the top of the head 140.

The socket 116 is configured for locking engagement of the fixation device 104, or more specifically, the insert 108 that at least partially surrounds the head 140 of the fixation device 104. In order to facilitate locking engagement, the surface of the socket 116 may include a rough or knurled surface and/or a surface fixation mechanism, such as ridges, grooves, bumps, pips, or the like to increase the coefficient of friction of the surface. In addition, the interior and exterior of the insert 108 as well as the head 140 may have rough or knurled surfaces and/or surface fixation mechanisms, such as ridges, grooves, bumps, pips, or the like to increase the coefficient of friction of the surfaces. For example, the surfaces may roughened by blasting, for example, with titanium oxide, glass beads or other suitable blasting material. One of skill in the art will understand that other surface treatments may also be used on the surfaces of the socket 116, the insert 108 and the head 140.

Thus, as the rod 106 is forced downward, such as by tightening of the locking element 112, the insert 108 and head 140 combination is forced downward into the socket 116, which preferably includes a lip 117. As shown, the lip 117 is tapered. More specifically, the lower hemisphere 156 of the insert 108 is compressed as it is forced into the socket 116 and lip 117 in a manner that forces a portion of the lower hemisphere 156 of the insert 108 both upward toward the top of the body 102 and inward toward the center of the insert 108. In turn, this may cause the insert 108 to exert forces on the lower hemisphere 148 of the head 140 that have both lateral and vertical components. Further, the interaction of the socket 116 and lip 117 with the insert 108 may cause the insert 108 to exert at least one force F2 upon the lower hemisphere 148 of the head 140 that is generally perpendicular to the surface of the upper hemisphere 146 of head 140 upon which the force F2 is exerted.

Thus, as the rod 106 is forced downward, the insert 108 preferably exerts forces on the upper hemisphere 146 of the head 140 that have both lateral and vertical components and forces on the lower hemisphere 148 of the head 140 that have both lateral and vertical components. Thus, the locking mechanism provides a more circumferential locking engagement than would otherwise result from the application of only vertical forces to the head 140. Moreover, the insert 108 may exert very specific forces having lateral and vertical components upon the head 140, including: a force F1 exerted upon the upper hemisphere 146 of the head 140 that is generally perpendicular to the surface of the upper hemisphere 146 of head 140 upon which the force F1 is exerted; and a force F2 exerted upon the lower hemisphere 148 of the head 140 that is generally perpendicular to the surface of the upper hemisphere 146 of head 140 upon which the force F2 is exerted.

Turning next to FIGS. 3A-3B, an alternate locking mechanism 300 is disclosed. The alternate locking mechanism 300 includes the same body 102, fixation device 104 and rod 106 disclosed in FIGS. 2A-B, but has alternate versions of the insert 108 and rod seat 110. The alternate insert 308 and rod seat 310 maintain the same reference numbering system as the insert 108 and rod seat 110 except that the reference numbers are provided in the 300s as opposed to of the 100s.

Accordingly, the insert 308 as shown in FIGS. 3A-B and 7A-B includes: upward extending fingers 350, downward extending fingers 352, upper hemisphere 354, lower hemisphere 356 and access hole 359. The insert 308 is similar to the insert 108 in design, material, form and function except that the insert 308 does not include a lip like the lip 158 of the insert 108. Similarly, the rod seat 310 as shown in FIGS. 6A-B and 8A-B includes: skirt 360, taper 362, bottom hole 363, taper 364, curvatures 366 and 368 and top hole 370. The rod seat 310 is similar to the rod seat 110 in design, material, form and function except that the rod seat 310 does not include a latch like the latch 172 of the rod seat 110. Otherwise, the locking mechanism 300 functions much the same as the locking mechanism 100.

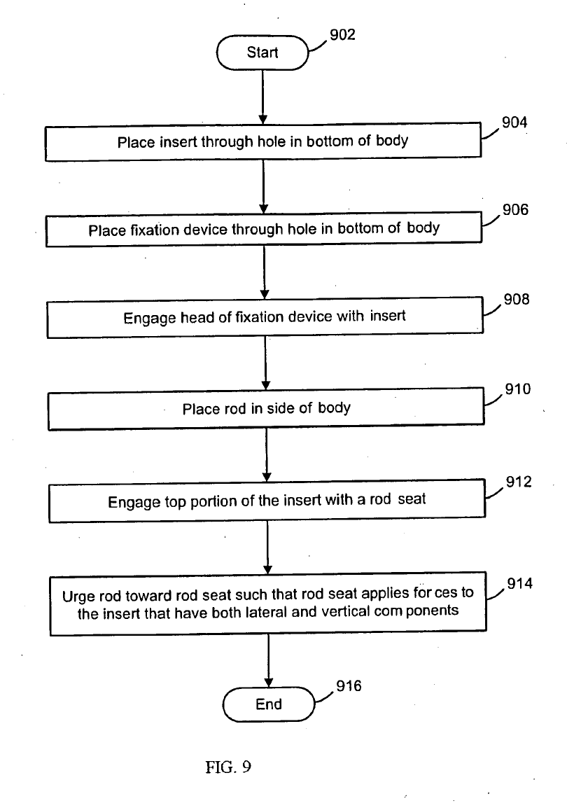

Turning next to FIG. 9, a flow chart illustrating a novel method of fixing the position of a rod with respect to a fixation device is provided. Flow begins at start block 902 from which progression continues to process block 904 wherein an insert, such as insert 108 or insert 308, is placed through a hole in the bottom of a body, such as body 102, of a locking mechanism. Progression then flows to process block 906 wherein a fixation device, such as the fixation device 140, is inserted through the hole in the bottom of the body. Progression then continues to process block 908 wherein the fixation device and insert are engaged.

Flow then progresses to process block 910 wherein a rod, such as the rod 106, is inserted into the side of the body. Progression then continues to process block 912 wherein a top portion of the insert is engaged with a rod seat located between the insert and the rod. Flow then continues to process block 914 wherein the rod is urged toward the rod seat such that the rod seat applies forces that have both lateral and vertical components to the insert. Flow then progresses to termination block 918.

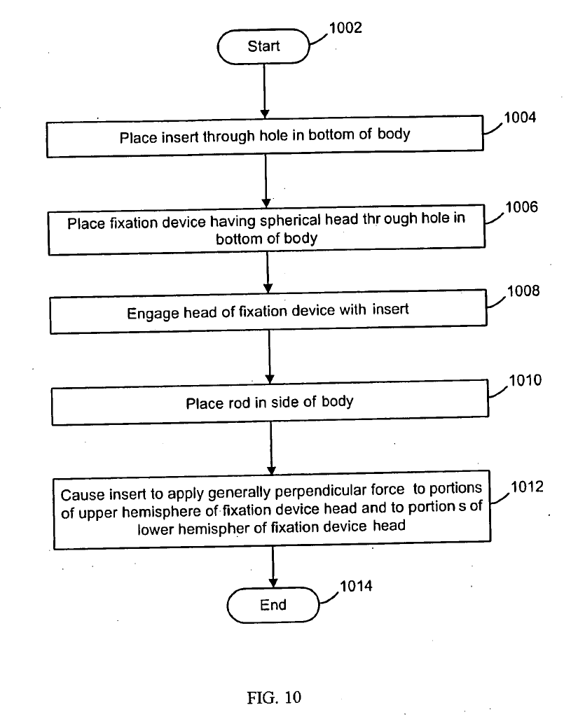

Turning next to FIG. 10, a flow chart illustrating an additional novel method of fixing the position of a rod with respect to a fixation device is provided. Flow begins at start block 1002 from which progression continues to process block 1004 wherein an insert, such as insert 108 or insert 308, is placed through a hole in the bottom of a body, such as body 102, of a locking mechanism. Progression then flows to process block 1006 wherein a fixation device having a spherical head, such as the fixation device 140, is inserted through the hole in the bottom of the body. Progression then continues to process block 1008 wherein the fixation device and insert are engaged.

Flow then progresses to process block 1010 wherein a rod, such as the rod 106, is inserted into the side of the body. Flow then continues to process block 1012 wherein the insert is caused to apply generally perpendicular force to portions of the upper hemisphere of the head of the fixation device and to portions of the lower hemisphere of the head of the fixation device. Flow then progresses to termination block 1014.

While the present invention has been described in association with exemplary embodiments, the described embodiments are to be considered in all respects as illustrative and not restrictive. Such other features, aspects, variations, modifications, and substitution of equivalents may be made without departing from the spirit and scope of this invention which is intended to be limited only by the scope of the following claims. Also, it will be appreciated that features and parts illustrated in one embodiment may be used, or may be applicable, in the same or in a similar way in other embodiments.

Although the invention has been shown and described with respect to certain embodiments, it is obvious that certain equivalents and modifications may be apparent to those skilled in the art upon the reading and understanding of the specification. The present invention includes all such equivalents and modifications, and is limited only by the scope of the following claims.

What is claimed is:

1. A locking mechanism comprising:
a body having a bottom portion and a side portion, the side portion being configured to receive a rod;
a fixation device extending at least partially through a hole in the bottom portion of the body, the fixation device comprising an at least partially spherical head;
an at least partially spherical insert at least partially surrounding the head of the fixation device; and
a rod seat having an opening for engaging a top portion of the insert such that upon engagement, the rod seat applies forces to the insert that have both lateral and vertical components.

2. The locking mechanism of claim 1 wherein the rod seat has a tapered portion for receiving the rod.

3. The locking mechanism of claim 2 wherein the center of the tapered portion of the rod seat is aligned with the central axis of the body.

4. The locking mechanism of claim 2 wherein the tapered portion of the rod seat is configured to engage any rod selected from a group of rods having diameters varying from 3 mm to 7 mm.

5. The locking mechanism of claim 1 wherein the fixation device is disengageable from the insert by pulling the insert toward the top of the body.

6. The locking mechanism of claim 1 wherein the rod seat comprises a skirt surrounding the opening for applying forces to the insert that have both lateral and vertical components.

7. The locking mechanism of claim 1 wherein the rod seat and the insert are snapably engageable.

8. The locking mechanism of claim 7 wherein the fixation device is disengageable from the insert by pulling the rod seat toward the top of the body.

9. The locking mechanism of claim 1 wherein linear compressive forces are not transferred from the rod seat to the insert along the central axis of the body.

10. The locking mechanism of claim 1 wherein linear compressive forces are not transferred from insert to the fixation device along the central axis of the body.

11. The locking mechanism of claim 1 wherein the insert is compressible such that the diameter of the insert is larger than the hole in the bottom portion of the body when the insert is in an uncompressed state and smaller than the diameter of the hole in the bottom portion of the body when the insert is in a compressed state.

12. The locking mechanism of claim 1 wherein the insert comprises:
downward fingers extending from a portion of the insert corresponding to an upper hemisphere of the head of the fixation device to a portion of the insert corresponding to a lower hemisphere of the head of fixation device; and
upward fingers extending from the portion of the insert corresponding to the lower hemisphere of the head of the fixation device to the portion of the insert corresponding to the upper hemisphere of the head of the fixation device.

13. The locking mechanism of claim 1 wherein the insert at least partially surrounds the head of the fixation device such that when the insert and fixation device are forced toward the bottom of the body, the insert applies generally perpendicular force to at least one location on a surface of an upper hemisphere of the head and further applies generally perpendicular force to at least one location on a surface of a lower hemisphere of the head.

14. A locking mechanism comprising:
a body having a bottom portion and a side portion, the side portion being configured to receive a rod;
a fixation device extending at least partially through a hole in the bottom portion of the body, the fixation device comprising an at least partially spherical head; and
an at least partially spherical insert at least partially surrounding the head of the fixation device such that when the insert and fixation device are forced toward the bottom of the body, the insert applies generally perpendicular force to at least one location on a surface of an upper hemisphere of the head of the fixation device and further applies generally perpendicular force to at least one location on a surface of a lower hemisphere of the head of the fixation device.

15. The locking mechanism of claim 14 further comprising a rod seat having a tapered portion for receiving the rod.

16. The locking mechanism of claim 15 wherein the center of the tapered portion of the rod seat is aligned with the central axis of the body.

17. The locking mechanism of claim 15 wherein the tapered portion of the rod seat is configured to engage any rod selected from a group of rods having diameters varying from 3 mm to 7 mm.

18. The locking mechanism of claim 15 wherein the rod seat has an opening for engaging a top portion of the insert and a skirt surrounding the opening for applying forces to the insert that have both lateral and vertical components.

19. The locking mechanism of claim 15 wherein the rod seat and the insert are snapably engageable.

20. The locking mechanism of claim 19 wherein the fixation device is disengageable from the insert by pulling the rod seat toward the top of the body.

21. The locking mechanism of claim 15 wherein linear compressive forces are not transferred from the rod seat to the insert along the central axis of the body.

22. The locking mechanism of claim 14 wherein linear compressive forces are not transferred from the insert to the fixation device along the central axis of the body.

23. The locking mechanism of claim 14 wherein the insert is compressible such that the diameter of the insert is larger than the hole in the bottom portion of the body when the insert is in an uncompressed state and smaller than the diameter of the hole in the bottom portion of the body when the insert is in a compressed state.

24. The locking mechanism of claim 14 wherein the insert comprises downward fingers and upward fingers.

25. The locking mechanism of claim 14 wherein the fixation device is disengageable from the insert by pulling the insert toward the top of the body.

26. A locking mechanism comprising:
a body having a bottom portion and a side portion, the side portion being configured to receive a rod selected from a group of rods of varying diameter;
a fixation device extending at least partially through a hole in the bottom portion of the body; and
a rod seat between the selected rod and the fixation device, the rod seat comprising a taper for engaging the selected rod and aligning the center of the selected rod with the central axis of the body the taper having two sides and at least two curvatures on each side, each curvature having a surface for engaging the selected rod such that the diameter of the selected rod determines which of the surfaces of the at least two curvatures engages the selected rod.

27. The locking mechanism of claim 26 further comprising an insert between the rod seat and a head of the fixation device.

28. The locking mechanism of claim 27 wherein the insert is at least partially spherical and at least partially surrounds the head of the fixation device.

29. The locking mechanism of claim 27 wherein the rod seat has an opening for engaging a top portion of the insert and a skirt surrounding the opening for applying forces to the insert that have both lateral and vertical components.

30. The locking mechanism of claim 27 wherein linear compressive forces are not transferred from the rod seat to the insert along the central axis of the body.

31. The locking mechanism of claim 27 wherein linear compressive forces are not transferred from insert to the fixation device along the central axis of the body.

32. The locking mechanism of claim 27 wherein the insert comprises downward fingers and upward fingers.

33. The locking mechanism of claim 27 wherein the fixation device is disengageable from the insert by pulling the insert toward the top of the body.

34. The locking mechanism of claim 27 wherein the rod seat and the insert are snapably engageable.

35. The locking mechanism of claim 34 wherein the fixation device is disengageable from the insert by pulling the rod seat toward the top of the body.

36. The locking mechanism of claim 26 wherein an upper portion of the taper of the rod seat is wider than a lower portion of the rod seat such that forcing the rod seat toward the bottom of the body forces the sides of the taper toward a central axis of the body.

37. The locking mechanism of claim 26 wherein the diameter of the selected rod varies from 3 mm to 7 mm.

38. The locking mechanism of claim 26 wherein the diameter of the selected rod causes the surfaces of at least two of the at least two curvatures to engage the selected rod.

39. The locking mechanism of claim 26 wherein the surfaces of at least two of the at least two curvatures overlap.

40. The locking mechanism of claim 26 wherein the surface of at least one curvature comprises a non-circular curve.

41-53. (canceled)
